(19) United States
(12) Patent Application Publication
(10) Pub. No.: US 2024/0024347 A1
(43) Pub. Date: Jan. 25, 2024

(54) PRODRUGS OF 6-MERCAPTOPURINE

(71) Applicants: The Johns Hopkins University, Baltimore, MD (US); Institute of Organic Chemistry and Biochemistry AS CR v.v.i., PRAGUE (CZ)

(72) Inventors: Barbara Slusher, Baltimore, MD (US); Kathryn Lemberg, Baltimore, MD (US); Rana Rais, Baltimore, MD (US); Pavel Majer, Prague (CZ); Marcela Krecmerova, Prague (CZ)

(21) Appl. No.: 18/042,971

(22) PCT Filed: Aug. 25, 2021
(86) PCT No.: PCT/US2021/047555

§ 371 Date: Feb. 24, 2023

Related U.S. Application Data
(60) Provisional application No. 63/069,876, filed on Aug. 25, 2020.

(51) Int. Cl. A61K 31/7076; C07H 19/207; A61P 35/02; A61K 31/606; A61K 31/573; A61K 39/395; A61K 31/519; A61K 31/475; A61K 31/7068; A61K 31/675; A61K 31/704

(52) U.S. Cl. CPC A61K 31/7076; C07H 19/207; A61P 35/02; A61K 31/606; A61K 31/573; A61K 39/3955; A61K 31/519; A61K 31/475; A61K 31/7068; A61K 31/675; A61K 31/704

(57) ABSTRACT

Prodrugs of 6-mercaptopurine (6-MP) and methods of their use for treating cancers and autoimmune diseases are disclosed.

### BACKGROUND

6-mercaptopurine (6-MP) is a drug that is widely used clinically to treat cancer and autoimmune disease. Specifically, 6-MP is used to treat acute lymphoblastic leukemia (ALL), chronic myeloid leukemia (CML), Crohn's disease, and ulcerative colitis. To be biologically active, 6-MP is biotransformed in human cells by a multistep enzymatic process that generates the active triphosphorylated nucleotide, 6-thioguanine nucleotide (6-TGN), which inhibits DNA synthesis. Several competing metabolic pathways methylate both 6-MP and its intermediary metabolites to generate inactive products that also cause liver toxicity (e.g., 6-methylmercaptopurine). Wide genetic variation in 6-mercaptopurine metabolism exists among patients, which leads to unnecessary side effects and limitations on 6-MP therapeutic dosing. Those with a genetic deficiency in thiopurine S-methyltransferase are at highest risk of side effects. These patients will require dose adjustments, especially for those with homozygous variant genotypes.

### SUMMARY

In some aspects, the presently disclosed subject matter provides a compound of formula (I):

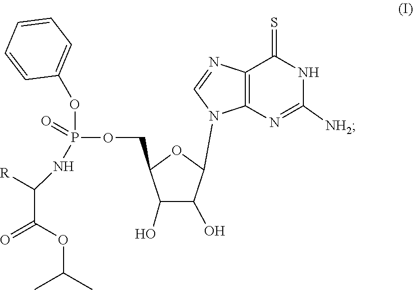

wherein R is C₁-C₄ straightchain or branched substituted or unsubstituted alkyl or heteroalkyl; and stereoisomers and pharmaceutically acceptable salts thereof.

In some aspects, R is a C₁-C₄ straightchain or branched substituted or unsubstituted alkyl or heteroalkyl group of an amino acid. In such aspects, R is selected from the group consisting of —CH₃ (alanine), —CH₂—CH(CH₃)₂ (leucine), —CH(CH₃)₂ (valine), —CH(CH₃)(CH₂CH₃) (isoleucine), —CH₂—OH (serine), —CH(OH)(CH₃) (threonine), —CH₂—SH (cysteine), —CH₂—CH₂—S—CH₃ (methionine), —CH₂—Ar, wherein Ar is phenyl (phenylalanine) or p-phenol (tyrosine).

In particular aspects, the compound of formula (I) is selected from the group consisting of:

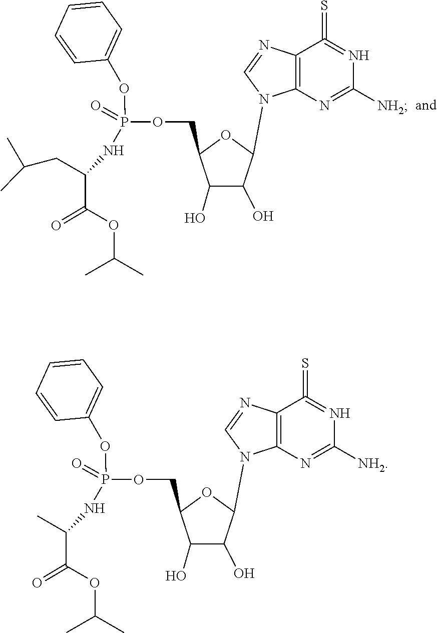

In yet more particular aspects, the compound of formula (I) is:

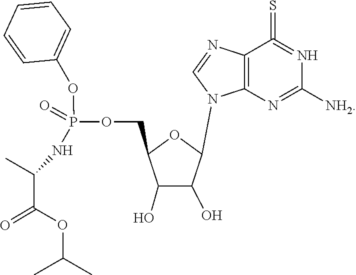

In some aspects, the presently disclosed subject matter provides a pharmaceutical formulation comprising a compound of formula (I).

In other aspects, the presently disclosed subject matter provides for the use of a compound of formula (I) for preparing a medicament.

In some aspects, the presently disclosed subject matter provides a method for treating a cancer or an autoimmune disease, the method comprising administering a therapeutically effective amount of a compound of formula (I) to a subject in need of treatment thereof.

In some aspects, the cancer is selected from the group consisting of acute lymphoblastic leukemia (ALL), chronic myeloid leukemia (CML), childhood acute lymphoblastic leukemia, acute promyelocytic leukemia (APL), acute myeloid leukemia (AML), and chronic myelomonocytic leukemia (CMML). In particular aspects, the cancer is acute lymphoblastic leukemia (ALL) or chronic myeloid leukemia (CML).

In some aspects, the cancer is a soft tissue sarcoma. In particular aspects, the soft tissue sarcoma is selected from the group consisting of a malignant peripheral nerve sheath tumor, a triton tumor, a rhabdomyosarcoma, and a synovial sarcoma.

In some aspects, the autoimmune disease is an inflammatory bowel disease. In particular aspects, the inflammatory bowel disease is Crohn's disease or ulcerative colitis.

In some aspects, the compound of formula (I) can be used to prevent organ transplant rejection.

In some aspects, the presently disclosed method further comprises administering one or more additional therapeutic agents with the compound of formula (I).

In some aspects, the disease is an inflammatory bowel disease and the one or more additional therapeutic agents is selected from the group consisting of mesalamine, prednisone, and infliximab.

In some aspects, the disease is acute lymphoblastic leukemia (ALL) and the one or more additional therapeutic agents is selected from the group consisting of methotrexate, vincristine, cytarabine, cyclophosphamide, daunorubicin, and prednisone.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE FIGURES

The patent or application file contains at least one drawing executed in color Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 is a diagram of 6-MP metabolism to active (6-TGTP) and inactive (6-MMP) products. Entry of novel phosphoramidate protide MK-905 into pathway is indicated. Based on known protide chemistry, MK905 is predicted to release 6-TGMP. The active form of 6-MP is 6-thioguanine triphosphate (6-TdGTP), which is generated by a multistep enzymatic biotransformation of 6-MP. Competing methylation reactions catalyzed by thiopurine methyltransferase (TPMT) and other enzymes prevent formation of 6TGTP and cause hepatotoxicity. There is significant variation among patients regarding the efficiency of 6-MP activation versus methylation;

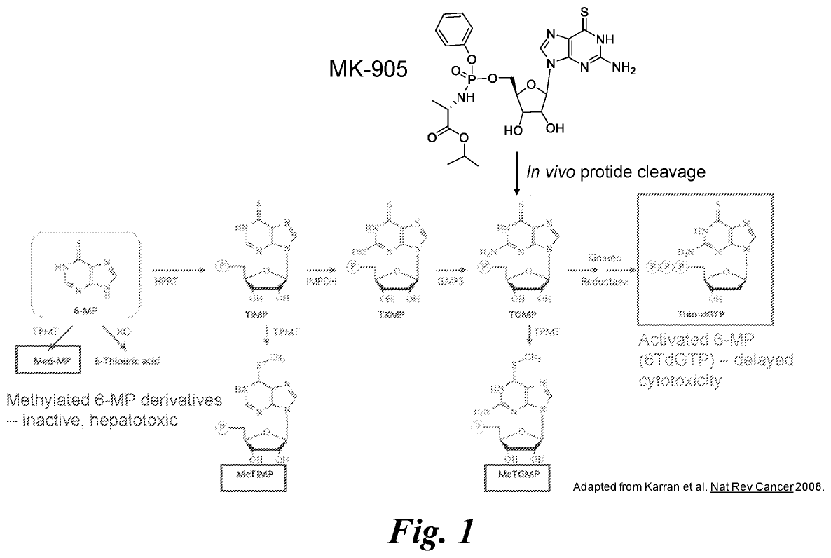

FIG. 2A and FIG. 2B demonstrate that single agent 6-MP partially inhibits growth of tumors in a murine sarcoma model in vivo with evidence for liver transaminase elevation. Following ten days of dosing with vehicle or 6-MP (20 mg/kg i.p.), 6-MP-treated mice had a mean tumor volume of 1,107 mm³, compared to vehicle-treated mice (2,038 mm³) (FIG. 2A). At sacrifice, blood ALT as a marker of liver inflammation was two-fold increased in 6-MP treatment mice compared to vehicle-treated mice (46 U/L vs 20 U/L) (FIG. 2B);

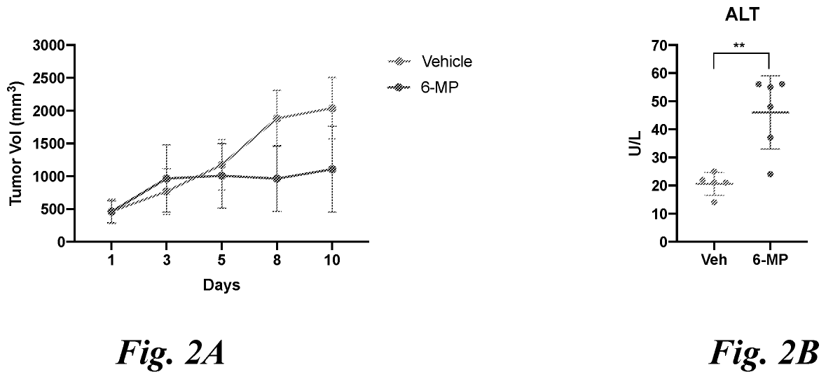

FIG. 3, panels A-D, demonstrates that single agent MK-905 inhibits growth of tumors in a murine sarcoma model in vivo without evidence for liver transaminase elevation. Following fourteen days of dosing with vehicle or MK-905 (13 mg/kg i.p.) MK-905 treated mice had a mean tumor volume of 415 mm³, compared to vehicle treated mice (1,657 mm³) (FIG. 3A). Tumor weights at sacrifice were significantly lower (359 mg) in MK-905 treated mice compared to vehicle treated mice (1276 mg) (FIG. 3B). Animal weights remained within 10% of starting weight between the two groups (FIG. 3C). Serum ALT as a marker of liver inflammation was normal and not significantly different between vehicle-treated and MK-905-treated mice (FIG. 3D);

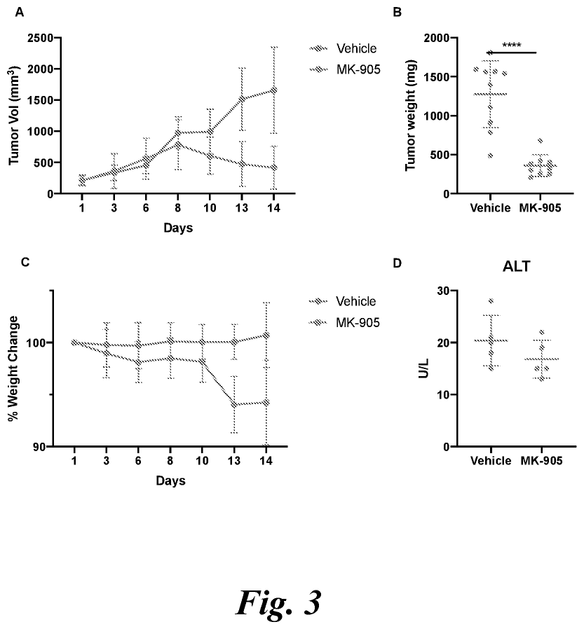

FIG. 4 shows the percent weight change in healthy female NSG mice (n=5/group) dosed with MK-905 (40 mg/kg i.p.×5 d/week or 20 mg/kg i.p.×5 d/week), 6-MP (20 mg/kg i.p.×5 d/week or 10 mg/kg i.p.×5 d/week), or vehicle;

FIG. 5 shows the whole body bioluminescence imaging (BLI) signal per treatment group in mice treated with MK-905, 6-MP, and vehicle; and

FIG. 6 shows the presence of active 6-TMGP metabolite in mouse plasma two hours following an initial dose of MK-905, 6-MP, and vehicle.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Prodrugs of 6-mercaptopurine

The presently disclosed subject matter addresses the barriers associated with treating cancers and autoimmune diseases with 6-mercaptopurine (6-MP). In some embodiments, the presently disclosed subject matter provides a prodrug of 6-MP. In particular embodiments, the prodrug of 6-MP is MK-905. MK-905 is a phosphoramidate prodrug of 6-MP that bypasses the need for three of the bioactivating steps and avoids the deleterious methylating side reactions that can limit 6-MP activity. In a murine tumor model, it was found that MK-905 inhibits tumor growth as a single agent at a lower dose than 6-MP, without evidence of liver toxicity. MK-905 therefore may be a novel antimetabolite tool for targeting cancer and autoimmune disease, such as IBD, and would be expected to allow lower dosing and fewer hepatotoxic side effects than 6-MP. Thus, the presently disclosed compounds inhibit cancer growth and inflammation in autoimmune disease and improve on the wide pharmacogenomic variation of 6-MP tolerability and efficacy. Accordingly, in some embodiments, the presently disclosed compounds can be used to treat cancers and autoimmune diseases including, but not limited to, acute lymphoblastic leukemia (ALL), chronic myeloid leukemia (CML), Crohn's disease, and ulcerative colitis.

A. Representative Compounds of Formula (I)

In some embodiments, the presently disclosed subject matter provides a compound of formula (I):

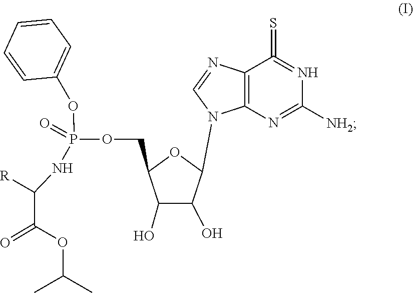

wherein R is C₁-C₄ straightchain or branched substituted or unsubstituted alkyl or heteroalkyl; and stereoisomers and pharmaceutically acceptable salts thereof.

As used herein, C₁-C₄ alkyl includes C₁, C₂, C₃, and C₄ alkyl, including methyl, ethyl, n-propyl, isopropyl, n-butyl, isobutyl, sec-butyl, and tent-butyl, each of which can be unsubstituted or substituted, for example, with hydroxyl or aryl. As also used herein, heteroalkyl includes a C₁-C₄ alkyl having at least one carbon atom substituted with a heteroatom, e.g., a sulfur atom.

In some embodiments, R is a C₁-C₄ straightchain or branched substituted or unsubstituted alkyl or heteroalkyl group of an amino acid. In such embodiments, R is selected from the group consisting of —CH₃ (alanine), —CH₂—CH(CH₃)₂ (leucine), —CH(CH₃)₂ (valine), —CH(CH₃)(CH₂CH₃) (isoleucine), —CH₂—OH (serine), —CH(OH)(CH₃) (threonine), —CH₂—SH (cysteine), —CH₂—CH₂—S—CH₃ (methionine), —CH₂—Ar, wherein Ar is phenyl (phenylalanine) or p-phenol (tyrosine).

In particular embodiments, the compound of formula (I) is selected from the group consisting of:

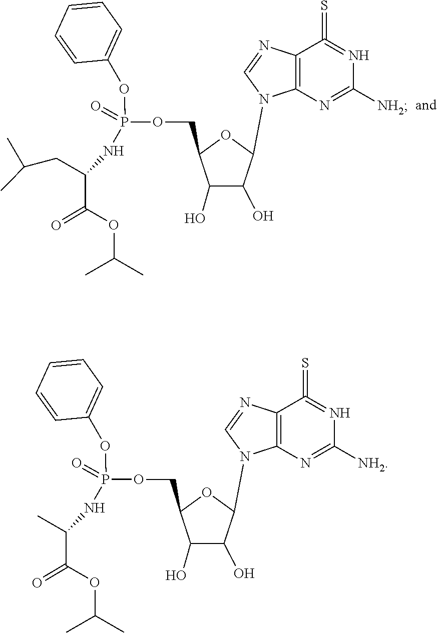

In yet more particular embodiments, the compound of formula (I) is:

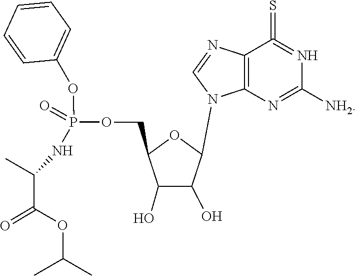

As provided herein below, in some embodiments, the presently disclosed subject matter provides a pharmaceutical formulation comprising a compound of formula (I).

In other embodiments, the presently disclosed subject matter provides for the use of a compound of formula (I) for preparing a medicament.

Representative prodrugs of 6-mercaptopurine are provided in Table 1.

TABLE 1
Structures of 6-Thioguanosine Prodrugs

| Cmpd. No. | Structure | MW |
|---|---|---|
| MK-901 | 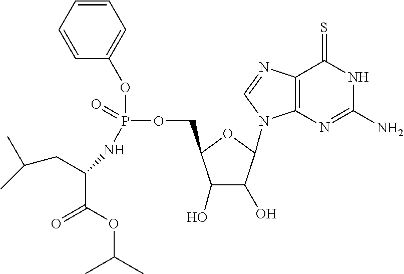 [SMILES: CC(C)C[C@H](NP(=O)(OC[C@H]1OC(n2cnc3c(=S)[nH]c(N)nc32)C(O)C1O)Oc1ccccc1)C(=O)OC(C)C] | 610.62 |
| MK-905 | 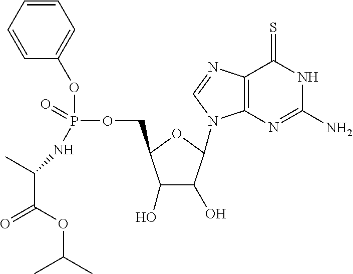 [SMILES: CC(C)OC(=O)[C@H](C)NP(=O)(OC[C@H]1OC(n2cnc3c(=S)[nH]c(N)nc32)C(O)C1O)Oc1ccccc1] | 568.54 |

B. Use of Compounds of Formula (I) for Treating Cancer and Autoimmune Disease

In some embodiments, the presently disclosed subject matter provides a method for treating a cancer or an autoimmune disease, the method comprising administering a therapeutically effective amount of a compound of formula (I) to a subject in need of treatment thereof:

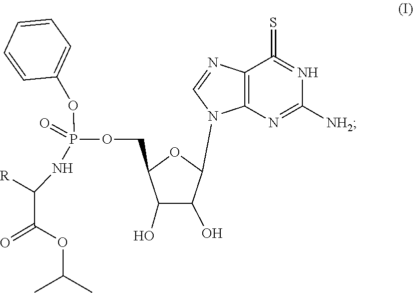

wherein R is C₁-C₄ straightchain or branched substituted or unsubstituted alkyl or heteroalkyl; and stereoisomers and pharmaceutically acceptable salts thereof.

In some embodiments, R is selected from the group consisting of —CH₃, —CH₂—CH(CH₃)₂, —CH(CH₃)₂, —CH(CH₃)(CH₂CH₃), —CH₂—OH, —CH(OH)(CH₃), —CH₂—SH, —CH₂—CH₂—S—CH₃, —CH₂—Ar, wherein Ar is phenyl or p-phenol.

In particular embodiments, the compound of formula (I) is selected from the group consisting of:

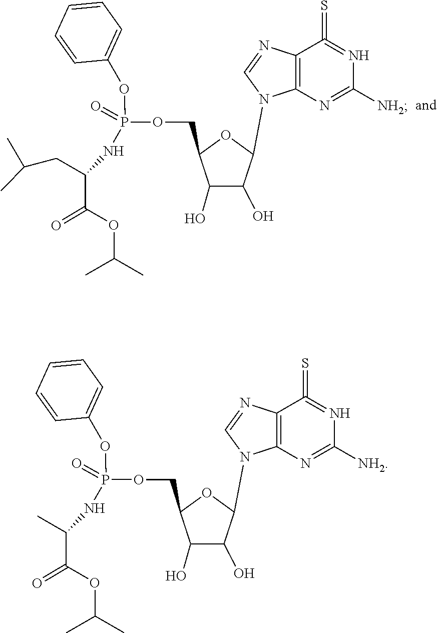

In yet more particular embodiments, the compound of formula (I) is:

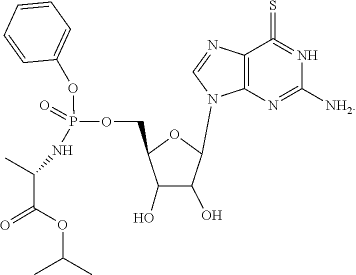

In some embodiments, the cancer is selected from the group consisting of acute lymphoblastic leukemia (ALL), chronic myeloid leukemia (CML), childhood acute lymphoblastic leukemia, acute promyelocytic leukemia (APL), acute myeloid leukemia (AML), and chronic myelomonocytic leukemia (CMML). in particular embodiments, the cancer is acute lymphoblastic leukemia (ALL) or chronic myeloid leukemia (CML).

In some embodiments, the cancer is a soft tissue sarcoma. In particular embodiments, the soft tissue sarcoma is selected from the group consisting of a malignant peripheral nerve sheath tumor, a triton tumor, a rhabdomyosarcoma, and a synovial sarcoma.

In some embodiments, the autoimmune disease is an inflammatory bowel disease. In particular embodiments, the inflammatory bowel disease is Crohn's disease or ulcerative colitis.

In some embodiments, the compound of formula (I) can be used to prevent organ transplant rejection.

In some embodiments, the presently disclosed method further comprises administering, one or more additional therapeutic agents with the compound of formula (1).

In some embodiments, the disease is an inflammatory bowel disease and the one or more additional therapeutic agents is selected from the group consisting of mesalamine, prednisone, and infliximab.

In some embodiments, the disease is acute lymphoblastic leukemia (ALL) and the one or more additional therapeutic agents is selected from the group consisting of methotrexate, vincristine, cytarabine, cyclophosphamide, daunorubicin, and prednisone.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed compound of formula (I), to block, partially block, interfere, decrease, or reduce the growth of a tumor or progression of an autoimmune disease. Thus, one of ordinary skill in the art would appreciate that the term “inhibit” encompasses a complete and/or partial decrease in the growth of the tumor or progression of the autoimmune disease, e.g., a decrease by at least 10%, in some embodiments, a decrease by at least 20%, 30%, 50%, 75%, 95%, 98%, and up to and including 100%.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound of formula (I) and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds of formula (I) described herein can be administered alone or in combination with adjuvants that enhance stability of the compounds of formula (I), alone or in combination with one or more other therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of formula (I) and at least one additional therapeutic agent can be varied so long as the beneficial effects of the combination of these agents are achieved. Accordingly, the phrase “in combination with” refers to the administration of a compound of formula (I) and at least one additional therapeutic agent either simultaneously, sequentially, or a combination thereof. Therefore, a subject administered a combination of a compound of formula (I) and at least one additional therapeutic agent can receive compound of formula (I) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of formula (I) and at least one additional therapeutic agent are administered simultaneously, they can be administered to the subject as separate pharmaceutical compositions, each comprising either a compound of formula (I) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of formula (I) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

C. Pharmaceutical Compositions and Administration

In another aspect, the present disclosure provides a pharmaceutical composition including one compound of formula (I) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000).

In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20th ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra -sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl- cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### II. Chemical Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position.

The substituents also may be further substituted (e.g., an aryl group substituent may have another substituent off it, such as another aryl group, which is further substituted at one or more positions).

Where substituent groups or linking groups are specified by their conventional chemical formula, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tent-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tent-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH=CH—O—CH₃, —Si(CH₃)₃, —CH₂—CH=N—OCH₃, —CH=CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity.

Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4- morpholinyl, 3-morpholinyl, tetrahydrofuran-2-yl, tetrahydrofuran-3-yl, tetrahydrothien-2-yl, tetrahydrothien-3-yl, 1 -piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-l-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); CH—CH═CH—CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, 0, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4- pyrimidyl, 5-benzothiazolyl, purinyl, 2-benzimidazolyl, 5-indolyl, 1-isoquinolyl, 5- isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

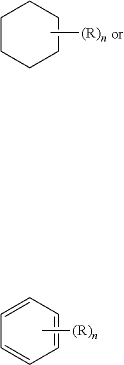

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

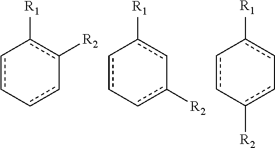

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g. , “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R—, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula —T—C(O)—(CRR)_{q}—U—, wherein T and U are independently —NR—, —O—, —CRR′— or a single bond, and q is an integer of from 0 to 3.

Alternatively, two of the substituents on adjacent atoms of aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described and include substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tent-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described. The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)- , or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups.

For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

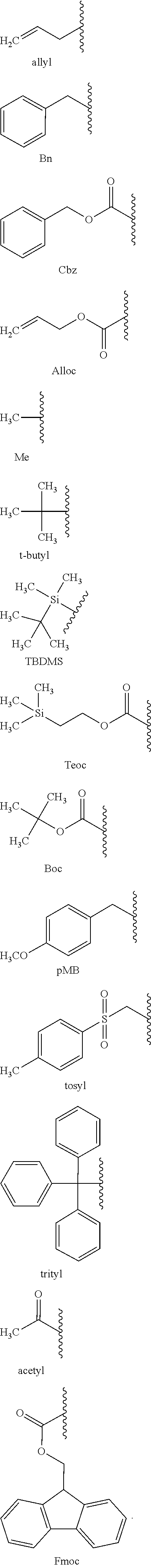

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### EXAMPLE 1

### Synthesis

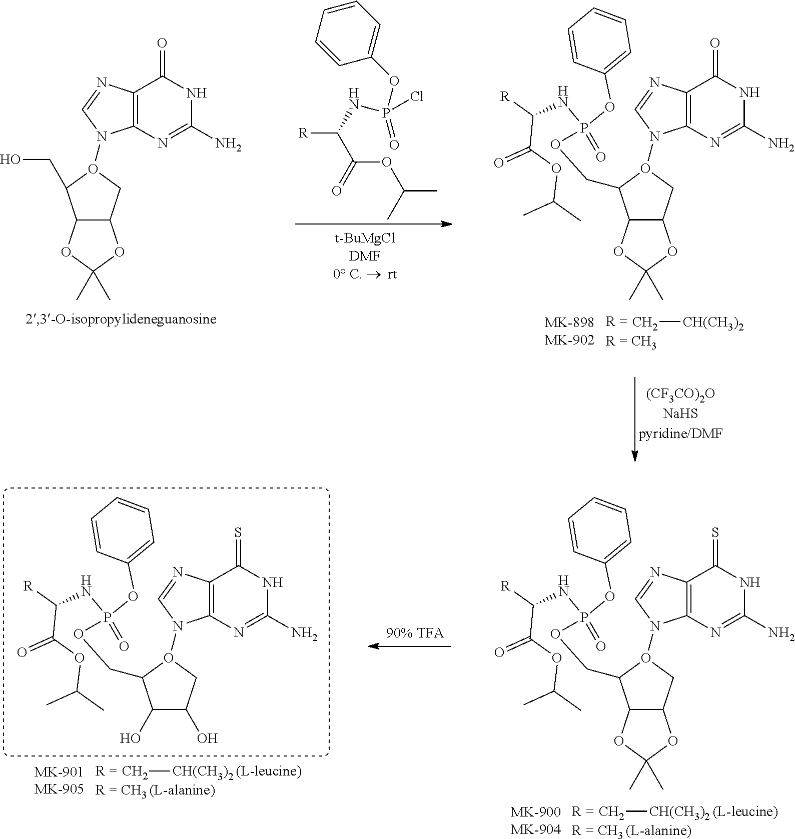

Synthesis of 2′,3′-O-isopropylideneguanosine was performed according to Foitzik et al., 2009. Amino acid phosphochloridates were synthesized from appropriate amino acid esters and phenyl phosphorodichloridate according to Sofia et al., 2010. The intermediate MK-902 is known from the literature, Ross et al., 2011, but for the purposes of the presently disclosed subject matter is prepared by a different way.

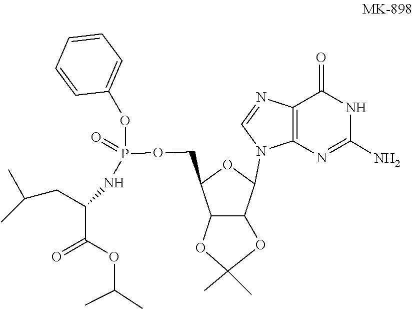 [SMILES: CC(C)C[C@H](NP(=O)(OC[C@H]1OC(n2cnc3c(=O)[nH]c(N)nc32)C2OC(C)(C)OC21)Oc1ccccc1)C(=O)OC(C)C]

A solution of 2′,3′-O-isopropylideneguanosine (500 mg; 1.55 mmol) in DMF (10 mL) was evaporated to ½ of the original volume and cooled to 0° C. tent-Butylmagnesium chloride (1M-solution in THF; 2.13 mL) was added under stirring. After 5 min, isopropyl [chloro(phenoxy)phosphoryl]-L-leucinate (862 mg; 2.48 mmol) was added. The mixture was stirred at 0° C. for 5 min and then for 2 h at room temperature (monitored by TLC in system chloroform-methanol 9:1). The solution was evaporated, the residue coevaporated with toluene and chromatographed on a column of silica gel (150 mL) in system chloroform-methanol (9:1). Yield: 670 mg (68%) of a white foam (2 diastereoisomers).
¹H NMR (DMSO-d₆, ppm) δ: 0.69-0.85 (m, 6H, CH₂—CH(CH₃)₂), 1.08-1.16 (m, 6H, O—CH(CH₃)₂), 1.29-1.65 (m, 9H, NHCH—CH₂—CH, CH₃ isopropylidene), 3.59-3.63 (m, 1H, CH—NH), 3.98-4.36 (m, 3H, H-4′, H-5′), 4.75-4.88 (m, 1H, O—CH(CH₃)₂), 5.10-5.15 (m, 1H, H-3′), 5.15-5.21 (m, 1H, H-2′), 5.89-6.01 (m, 1H, P—NH), 6.01-6.06 (m, 1H, H-1′), 6.55 (bs, 2H, NH₂), 7.09-7.21 (m, 3H, H-2″, H-4″), 7.28-7.39 (m, 2H, H-3″), 7.85 (s, 1H, H-8), 10.67 (bs, 1H, NH).
ESIMS, m/z: 657.2 (M+Na)⁺ (100), 635.3 (MH)⁺ (6).
HRMS (ESI): For C₂₈H₃₉O₉N₆NaP (M+Na)⁺ calculated: 657.24083; found: 657.24092. For C₂₈H₄₀O₉N₆P (MH)⁺ calculated: 635.25869; found: 635.25916.

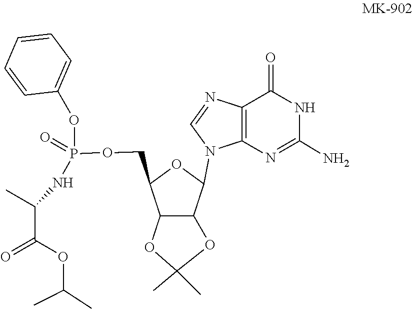 [SMILES: CC(C)OC(=O)[C@H](C)NP(=O)(OC[C@H]1OC(n2cnc3c(=O)[nH]c(N)nc32)C2OC(C)(C)OC21)Oc1ccccc1]

A solution of 2′,3′-O-isopropylideneguanosine (500 mg; 1.55 mmol) in DMF (10 mL) was evaporated to ½ of the original volume. The cloudy solution was cooled to 0° C. and tent-butylmagnesium chloride (1M-5 solution in THF; 2.13 mL) was added under stirring. After 5 min, isopropyl [chloro(phenoxy)phosphoryl]-L-alaninate (758 mg; 2.48 mmol) was added as a solution in DMF (2 mL). The mixture was stirred at 0° C. for 5 min and then for 24 h at room temperature (monitored by TLC in system chloroform—methanol 9:1). The reaction mixture was evaporated, the residue coevaporated with toluene and chromatographed on a column of silica gel (150 mL) in system chloroform—methanol ((95:5), elution of by-products), followed by chloroform—methanol ((9:1), elution of product). Yield: 580 mg (63%) of a white amorphous solid (2 diastereoisomers).
¹H NMR (DMSO-d₆, ppm) δ: 1.08-1.21 (m, 9H, CH₃—CH—NH, CH(CH₃)₂), 1.29-1.36 (m, 3H, CH₃ isopropylidene), 1.49-1.54 (m, 3H, CH₃ isopropylidene), 3.67-3.83 (m, 1H, CH—NH), 4.00-4.37 (m, 3H, H-4′, H-5′), 4.78-4.88 (m, 1H, OCH(CH₃)₂), 5.10-5.16 (m, 1H, H-3′), 5.16-5.21 (m, 1H, H-2′), 5.96-6.05 (m, 2H, H-1′, P—NH), 6.55 (bs, 2H, NH₂), 7.07-7.23 (m, 3H, H-2″, H-4″), 7.28-7.41 (m, 2H, H-3″), 7.85 (s, 1H, H-8), 10.71 (bs, 1H, NH).
ESIMS, m/z: 615.2 (M+Na)⁺ (100), 593.2 (MH)⁺ (7).
HRMS (ESI): For C₂₅H33O₉N₆NaP (M+Na)⁺ calculated: 615.19388; found: 615.19391. For C₂₅H₃₄O₉N₆P (MH)⁺ calculated: 593.21194; found: 593.21211.
2″, 3″-O-Isopropylidene 6-thioguanosine proTides.

### General Procedure for MK-900 and MK-904

Trifluoroacetic anhydride (0.6 mL; 4.3 mmol) was added dropwise under argon to a 0° C. cold solution of guanosine derivative MK-898 or MK-892 (0.5 mmol) in dry pyridine (10 mL). The mixture was removed from ice bath and stirred at room temperature for 40 min. After that, a suspension of dry NaHS (0.87 g; 5 mmol) in DMF (15 mL) was added (dissolution occurred). The mixture was stirred at room temperature till disappearance of starting compound (TLC control in system 5% MeOH in CHCl₃). The mixture was evaporated and the residue chromatographed on a column of silica gel (150 mL; short wide column) in a gradient of methanol in chloroform (0-5%). The crude product was eluted by 5% MeOH in CHCl₃, together with colored (purple) impurities. Final purification of the product was performed by additional chromatography on silica gel (50 mL) under the same conditions.

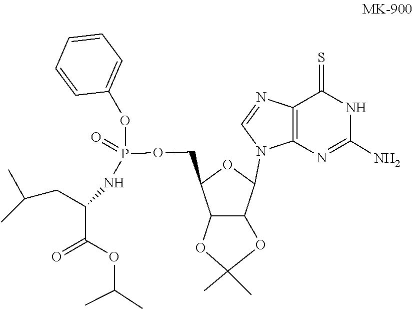 [SMILES: CC(C)C[C@H](NP(=O)(OC[C@H]1OC(n2cnc3c(=S)[nH]c(N)nc32)C2OC(C)(C)OC21)Oc1ccccc1)C(=O)OC(C)C]

Reaction time with NaHS: 20 h. Yield: 186 mg (57%) of a white foam (2 diastereoisomers).
¹H NMR (DMSO-d₆, ppm) δ: 0.71-0.82 (m, 6H, CH₂—CH(CH₃)₂), 1.09-1.13 (m, 6H, O—CH(CH₃)₂), 1.31-1.64 (m, 9H, NHCH—CH₂ —CH, CH₃ isopropylidene), 3.60-3.68 (m, 1H, CH—NH), 3.98-4.35 (m, 3H, H-4′, H-5′), 4.75-4.87 (m, 1H, O—CH(CH₃)₂), 5.11-5.14 (m, 1H, H-3′), 5.18-5.20 (m, 1H, H-2′), 5.91-6.03 (m, 1H, P—NH), 6.05 (m, 1H, H-1′), 6.87 (bs, 2H, NH₂), 7.10-7.17 (m, 3H, H-2″, H-4″), 7.30-7.36 (m, 2H, H-3″), 8.03 (s, 1H, H-8), 12.02 and 12.04 (2× bs, 1H, NH).
¹³C NMR (DMSO-d₆, ppm) δ: 23.90 and 24.06 (CH₂—CH(CH₃)₂), 21.31-22.83 (m, CH(CH₃)₂), 25.43, 25.45, 27.15 and 27.17 (CH₃isopropylidene), 42.07-42.28 (m, CH-CH₂—CH), 52.89 and 55.03 (CH—NH), 65.98-66.05 (m, C-5′), 68.09 and 68.12 (O—CH(CH₃)₂), 81.15 and 81.24 (C-3′), 83.81 and 83.90 (C-2′), 85.24-85.54 (m, C-4′), 88.74 and 88.91 (C-1′), 113.44 and 113.46 (O—C—O), 120.04-120.31 (m, C-2″), 124.68 and 124.74 (C-4″), 128.75 and 128.77 (C-5), 129.68 and 129.77 (C-3″), 139.07 and 139.20 (C-8), 147.12 and 147.20 (C-4), 150.75-150.86 (m, C-1″), 153.14 (C-2), 172.84-173.07 (m, COO), 175.57 and 175.58 (C-6).
ESIMS, m/z: 673.2 (M+Na)⁺ (100), 651.3 (MH)⁺ (60).
HRMS (ESI): For C₂₈H₃₉O₈N₆NaPS (M+Na)⁺ calculated: 673.21799; found: 673.21808. For C₂₈H40O₈N₆PS (MH)⁺ calculated: 651.23605; found: 651.23622.

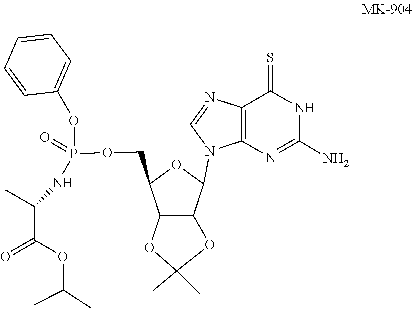 [SMILES: CC(C)OC(=O)[C@H](C)NP(=O)(OC[C@H]1OC(n2cnc3c(=S)[nH]c(N)nc32)C2OC(C)(C)OC21)Oc1ccccc1]

Reaction time with NaHS: 2 h. Yield: 167 mg (55%) of a white foam (2 diastereoisomers).
¹H NMR (DMSO-d₆, ppm) δ: 1.10-1.17 (m, 9H, CH₃—CH—NH, CH(CH₃)₂), 1.31-1.52 (m, 6H, CH₃ isopropylidene), 3.69-3.78 (m, 1H, CH—NH), 3.99-4.36 (m, 3H, H-4′, H-5′), 4.76-4.86 (m, 1H, OCH(CH₃)₂), 5.11-5.15 (m, 1H, H-3′), 5.18-5.21 (m, 1H, H-2′), 5.96-6.05 (m, 2H, H-1′, P—NH), 6.86 (bs, 2H, NH₂), 7.10-7.19 (m, 3H, H-2″, H-4″), 7.30-7.37 (m, 2H, H-3″), 8.03 (s, 1H, H-8), 12.02 (bs, 1H, NH).
¹³C NMR (DMSO-d₆, ppm) δ: 19.74-19.86 (m, NH—CH—CH₃), 21.56-21.61 (m, CH₃ isopropyl), 25.46, 25.49, 27.18 and 27.20 (CH₃ isopropylidene), 49.94 and 50.12 (CH—NH), 65.92 and 66.01 (2×d, J_{C,P}=5.1, C-5′), 68.20 (O—CH(CH₃)₂), 81.18 (C-3′), 83.83 and 83.92 (C-2′), 85.28 and 85.53 (2×d, J_{C,P}=8.0 and 7.9, C-4′), 88.82 and 88.92 (C-1′), 113.47 and 113.49 (O—C—O), 120.25-120.33 (m, C-2″), 124.76 and 15 124.81 (C-4″), 128.76 and 128.79 (C-5), 129.77 and 129.83 (C-3″), 139.11 and 139.24 (C-8), 147.17 and 147.24 (C-4), 150.79 (d, J_{C,P}=6.3, C-1″), 153.18 (C-2), 172.81 (d, J_{C,P}=4.5, COO), 172.92 (d, J_{C,P}=4.2, COO), 175.58 (C-6).
ESIMS, m/z: 631.1 (M+Na)⁺ (100), 609.1 (MH)⁺ (6).
HRMS (ESI): For C₂₅H₃₃O₈N₆NaPS (M+Na)⁺ calculated: 631.17104; found: 631.17129. For C₂₅H₃₄O₈N₆PS (MH)⁺ calculated: 609.18910; found: 609.18944.
Deprotection of Isopropylidene Group. General Procedure for 6-thioguanosine proTides MK-901 and MK-905.
90% Trifluoroacetic acid (10 mL) was added to isopropylidene derivative MK-900 or MK-904 (0.2 mmol). The solution was stirred for 20 min at room temperature and evaporated. The residue was coevaporated with toluene (2×20 mL) and chromatographed on o column of silica gel (20 mL) in system methanol—chloroform (9:1). Final purification of the crude product was performed by additional chromatography in system ethyl acetate—acetone—ethanol—water (43:3:2:2). Appropriate fractions were evaporated and the residue lyophilized from aqueous acetonitrile or crystallized from a mixture acetonitrile—acetone (1:1).

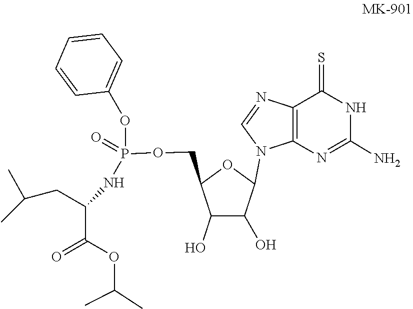 [SMILES: CC(C)C[C@H](NP(=O)(OC[C@H]1OC(n2cnc3c(=S)[nH]c(N)nc32)C(O)C1O)Oc1ccccc1)C(=O)OC(C)C]

Yield: 75 mg (61%) of a white lyophilizate (2 diastereoisomers).
¹H NMR (DMSO-d₆, ppm) δ: 0.69-0.82 (m, 6H, CH₂—CH(CH₃ )₂), 1.10-1.15 (m, 6H, OCH(CH₃)₂), 1.35-1.66 (m, 3H, NH—CH—CH₂—CH), 3.61-3.71 (m, 1H, CH—NH), 4.00-4.25 (m, 4H, H-3′, H-4′, H-5′), 4.39-4.44 (m, 1H, H-2′), 4.77-4.89 (m, 1H, O—CH(CH₃)₂), 5.32 and 5.35 (2×d, 1H, J_{OH,3′}=5.0 and 4.8, 3′-OH), 5.56-5.57 (m, 1H, 2′-OH), 5.71 and 5.73 (2×d, 1H, J_{1′,2′}=5.9 and 6.0, H-1″), 5.92-6.05 (m, 1H, P—NH), 6.83 (bs, 2H, NH₂), 7.14-7.19 (m, 3H, H-2″, H-4″), 7.31-7.37 (m, 2H, H-3″), 8.03 and 8.04 (2×s, 1H, H-8), 11.97 (bs, 1H, NH).
¹³C NMR (DMSO-d₆, ppm) δ: 21.35-22.80 (m, CH₃), 23.92 and 24.08 (CH₂-CH(CH₃)₂), 42.16 and 42.39 (2×d, J_{C,P}=8.1 and 7.3, CH—CH₂—CH), 52.96 and 53.09 (CH—NH), 66.23 and 66.36 (2×d, J_{C,P}=5.1 and 5.3, C-5′), 68.11 and 68.15 (O—CH(CH₃)₂), 70.36 and 70.43 (C-3′), 73.27 and 73.36 (C-2′), 82.74-82.91 (m, C-4′), 86.58 and 86.62 (C-1″), 120.08 and 120.39 (2×d, J_{C,P}=4.9 and 4.7, C-2″), 124.66 and 124.75 (C-4″), 128.54 and 128.56 (C-5), 129.69 and 129.78 (C-3″), 138.40 and 138.49 (C-8), 148.17 (C-4), 150.82-150.96 (m, C-1″), 153.28 (C-2), 172.88-173.17 (m, COO), 175.36 (C-6).
ESIMS, m/z: 633.2 (M+Na)⁺ (100), 611.2 (MH)⁺ (55).
HRMS (ESI): For C₂₅H₃₅O₈N₆NaPS (M+Na)⁺ calculated: 633.18669; found: 633.18676. For C₂₅H₃₆O₈N₆PS (MH)⁺ calculated: 611.20475; found: 611.20481.

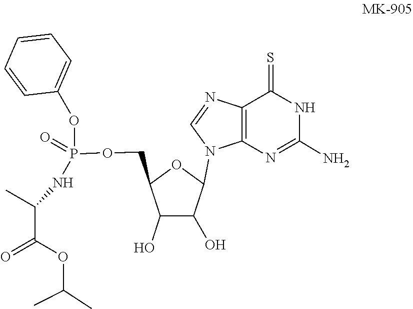 [SMILES: CC(C)OC(=O)[C@H](C)NP(=O)(OC[C@H]1OC(n2cnc3c(=S)[nH]c(N)nc32)C(O)C1O)Oc1ccccc1]

Yield: 69 mg (61%) of white crystals (2 diastereoisomers).
¹H NMR (DMSO-d₆, ppm) δ: 1.12-1.21 (m, 9H, CH₃), 3.70-3.82 (m, 1H, CH—NH), 4.04-4.27 (m, 4H, H-3′, H-4′, H-5′), 4.41-4.44 (m, 1H, H-2′), 4.79-4.88 (m,1H, CH(CH₃)₂), 5.32-5.36 (m, 1H, 3′-OH), 5.56-5.58 (m, 1H, 2′-OH), 5.72 and 5.73 (2×d, 1H, J_{1′, 2′}=6.0 and 5.9, H-1″), 5.99-6.06 (m, 1H, P—NH), 6.82 (bs, 2H, (NH₂), 7.14-7.22 (m, 3H, H-2″, H-4″), 7.32-7.38 (m, 2H, H-3″), 8.03 and 8.04 (2×s, 1H, H-8), 11.98 (bs, 1H, NH).
¹³C NMR (DMSO-d₆, ppm) δ: 19.80 and 19.93 (2×d, J_{C,P}=7.3 and 6.6, CH₃—CHNH), 21.56-21.62 (m, CH₃ isopropyl), 49.98 and 50.14 (CH—NH), 66.10 and 66.21 (2×d, J_{C,P}=4.9 and 5.2, C-5′), 68.19 (CH isopropyl), 70.37 and 70.39 (C-3′), 73.28 and 73.36 (C-2′), 82.76-82.83 (m, C-4′), 86.58 and 86.69 (C-1″), 120.31 and 120.32 (2×d, J_{C,P}=4.8, C-2″), 124.72 and 124.75 (C-4″), 128.52 and 128.54 (C-5), 129.75 and 129.80 (C-3″), 138.39 and 138.47 (C-8), 148.14 and 148.17 (C-4), 150.83 and 150.86 (2×d, J_{C,P}=6.2 and 6.3, C-1″), 153.28 (C-2), 172.81 and 172.93 (2×d, J_{C,P}=4.9 and 4.3, COO), 175.35 and 175.36 (C-6).
ESIMS, m/z: 591.1 (M+Na)⁺ (100), 569.1 (MH)⁺ (8).
HRMS (ESI): For C₂₂H₂₉O₈N₆NaPS (M+Na)⁺ calculated: 591.13974; found: 591.13983.
For C₂₂H₃₀O₈N₆PS (MH)⁺ calculated: 569.15780; found: 569.15796.
Structure numbering for NMR assignments:

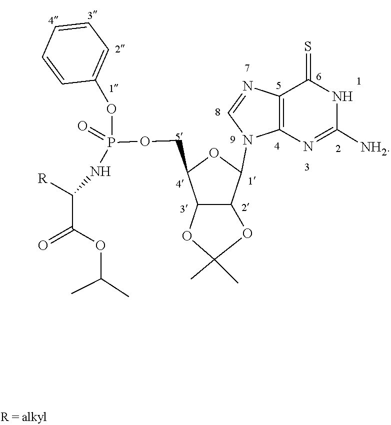

### EXAMPLE 2

### Methods

Experimental procedures were approved by the Johns Hopkins University Animal Care and Use Committee under the protocol #M019M213.

C₅₇BL/6 mice at 10 weeks of age between 25 g and 30 g were obtained from Envigo. To induce solid tumor growth in animals, 3-5 million murine NPcis (NF1^{+/-};p53^{+/-}) tumor cells grown and harvested from cell culture, were injected subcutaneously to the right flank. Tumors were palpable at the site of injection between 17 to 22 days post-injection. Animals were then randomly assigned to a treatment arm: Vehicle or MK-905 (n=10/group; average tumor volume of ˜200 mm³). The treatment regimen was as follows: IP vehicle daily or IP MK905 daily (13 mg/kg/dose). Animals were treated for 14 days (MK-905 study). Tumor volume was calculated by the formula: volume=(L×(W²))/2. Weights and tumor volumes were measured three times weekly; tumor and blood were harvested on day 14. In a separate experiment, a similar protocol employing the same mouse model was used to explore antitumor effects of IP 6-mercaptopurine (6MP; 20 mg/kg/dose daily 5 days on/2 days off) compared to IP vehicle. The average tumor volume in this study was ˜460 mm³ with n=7/group. This study was run for 10 days, terminated on day 11 due in part to volume of tumors in vehicle group.

### EXAMPLE 3

### Evaluation of MK-905 in a Mouse Model of Acute Lymphoblastic Leukemia

### 3.1 Overview

This example provides data evaluating MK-905 in a mouse model of acute lymphoblastic leukemia. These data demonstrate the efficacy of MK-905 in acute lymphoblastic leukemia, with a tolerability profile that may be better than the currently used 6-mercaptopurine.

### 3.2 Data

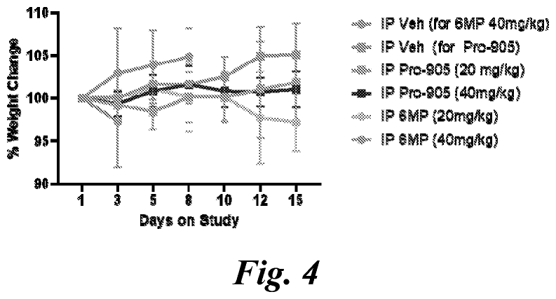

Tolerability of MK-905 compared to 6-MP was evaluated in healthy NSG mice. Female NSG mice (n=5/group) were dosed with MK-905 (40 mg/kg i.p.×5 d/week or 20 mg/kg i.p.×5 d/week), 6-MP (20 mg/kg i.p.×5 d/week or 10 mg/kg i.p.×5 d/week), or vehicle. Animal weights and body condition scores were measured three times per week. After twenty one days, surviving mice were euthanized and blood collected for clinical chemistry measurements (complete blood count with differential, clinical chemistries). As summarized in Table 2, 6-MP at 40 mg/kg was toxic to mice with no mice surviving >2 weeks. 80% of mice survived >2 weeks in the MK-905 40 mg/kg group, and 100% of mice survived the study in the vehicle and MK-905 20 mg/kg groups. Myelosuppression (decreased white blood cell count and platelet count compared to vehicle) was noted in the MK-905 40 mg/kg and 6-MP 20 mg/kg groups at study termination. No weight loss >10% was noted in any of the groups (FIG. 4).

TABLE 2
Comparison of the Efficacy of MK-905 and 6MP a
Mouse Model of Acute Lymphoblastic Leukemia.

| | | Myelosuppression |
|---|---|---|
| Dose (5 | | (marker for active |
| d/week, i.p.) | Mice surviving >2 weeks | metabolite) |
| Vehicle | 5/5 | control |
| MK-905 (40 mg/kg) | 4/5 | Y |
| MK-905 (20 mg/kg) | 5/5 | N |
| 6MP (40 mg/kg) | 0/5 | n/a |
| 6MP (20 mg/kg) | 4/5 | Y |

Based on these results the recommended dose for MK-905 in an efficacy study in a mouse model of B-cell lymphoblastic leukemia was: 40 mg/kg i.p per day (study days 3-7), 20 mg/kg i.p./day (days 10-14, 17-21). 5days/week. 6-MP was also evaluated in comparison at 20 mg/kg i.p. per day (study days 3-7), 10 mg/kg i.p. per day (days 10-14, 17-21). The molar equivalent dose of MK-905 used was 40% lower than that of 6-MP.

The in vivo antitumor efficacy of MK-905 was evaluated in the NALM6-Luc-mCh-Puro human acute lymphoblastic leukemia in female NSG mice. Mice were inoculated with tumor cells on day 0, staged by BLI on day 3, and sorted into matched treatment groups. The primary endpoint of the study was tumor bioluminescence imaging (BLI) signal. Secondary endpoints included: animal survival, weight changes, and clinical pathology markers. Single timepoint drug metabolite testing was conducted after the first dose of MK-905. Studies were run in comparison to vehicle or 6-MP treatment.

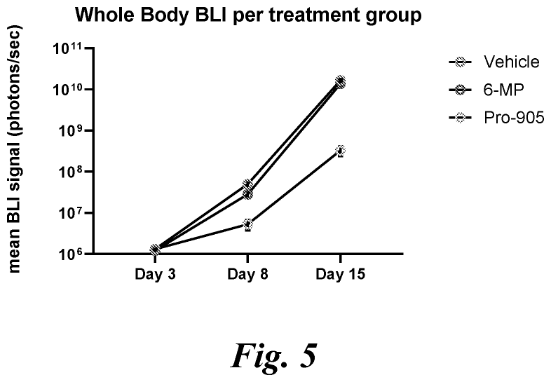

The day 15 mean BLI signal in the MK-905 treated mice (n=10) was 3.27×10⁸ photons/sec, compared with 1.65×10¹⁰ photons/sec in the vehicle treated group and 1.36×10¹⁰ photons/sec in the 6-MP-treated group. MK-905 treated mice had 1.3% of the median vehicle group tumor BLI signal on day 15 (FIG. 5). 6-MP treated mice by comparison had 77% of the median vehicle group tumor BLI signal on day 15. These data demonstrate that at a lower molar equivalent dose, MK-905 is more efficacious in a B-ALL mouse model.

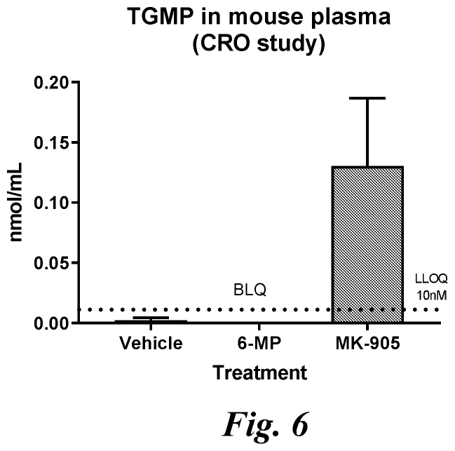

At two hours following initial dose of MK-905, conversion to the active 6-thioguanine monophosphate (6-TGMP) metabolite was observed, whereas no active metabolite was observed at this time point following 6-MP dosing (FIG. 6). The chemical structure of 6-TGMP is provided herein below:

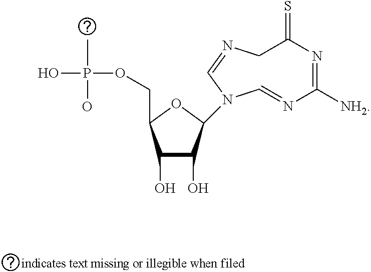

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Foitzik, R. C.; Devine, S. M.; Hausler, N. E.; Scammells, P. J. Linear and convergent approaches to 2-substituted adenosine-5′-N-alkylcarboxamides. Tetrahedron 2009, 65, 8851-8857.
Sofia, M. J.; Bao, D.; Chang, W.; Du, J.; Nagarathnam, D.; Rachakonda, S.; Reddy, P. G.; Ross, B. S.; Wang, P.; Zhang, H.-R.; Bansal, S.; Espiritu, C.; Keilman, M.; Lam, A. M.; Steuer, H. M. M.; Niu, C.; Otto, M. J.; Furman, P. A. Discovery of a β-D-2′-Deoxy-2′-α-fluoro-2′-β-C-methyluridine Nucleotide Prodrug (PSI-7977) for the Treatment of Hepatitis C Virus. J. Med. Chem. 2010, 53, 7202-7218.
Ross, B. S.; Reddy, P. G.; Zhang, H.-R.; Rachakonda, S.; Sofia, M. J. Synthesis of Diastereomerically Pure Nucleotide Phosphoramidates. J. Org. Chem. 2011, 76, 8311-8319.

Although the foregoing subject matter has been described in some detail by way of illustration and example for purposes of clarity of understanding, it will be understood by those skilled in the art that certain changes and modifications can be practiced within the scope of the appended claims.

1. A compound of formula (I): 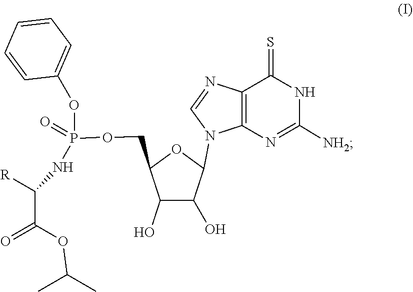
wherein R is C₁-C₄ straightchain or branched substituted or unsubstituted alkyl or heteroalkyl; and
stereoisomers and pharmaceutically acceptable salts thereof.

2. The compound of claim 1, wherein R is selected from the group consisting of —CH₃, —CH₂—CH(CH₃)₂, —CH(CH₃)₂, —CH(CH₃)(CH₂CH₃), —CH₂—OH, —CH(OH)(CH₃), —CH₂—SH, —CH₂—CH₂—S—CH₃, —CH₂—Ar, wherein Ar is phenyl or p-phenol.

3. The compound of claim 1, wherein the compound of formula (I) is selected from the group consisting of:

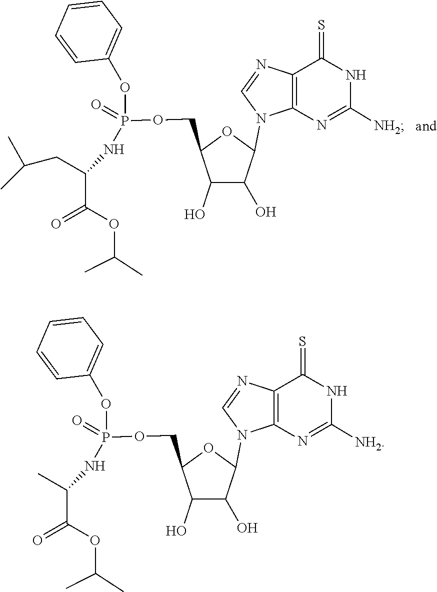

4. The compound of claim 3, wherein the compound of formula (I) is:

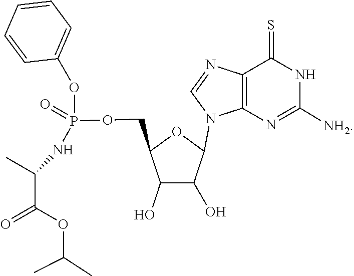 [SMILES: CC(C)OC(=O)[C@H](C)NP(=O)(OC[C@H]1OC(n2cnc3c(=S)[nH]c(N)nc32)C(O)C1O)Oc1ccccc1]

5. A pharmaceutical formulation comprising a compound of any of claims 1.

6. (canceled)

7. A method for treating a cancer or an autoimmune disease, the method comprising administering a therapeutically effective amount of a compound of formula (I) to a subject in need of treatment thereof: 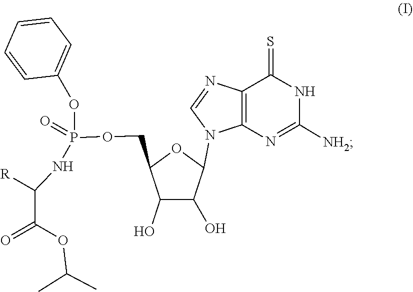
wherein R is C₁-C₄ straightchain or branched substituted or unsubstituted alkyl or heteroalkyl; and
stereoisomers and pharmaceutically acceptable salts thereof.

8. The method of claim 7, wherein R is selected from the group consisting of —CH₃, —CH₂—CH(CH₃)₂, —CH(CH₃)₂, —CH(CH₃)(CH₂CH₃), —CH₂—OH, —CH(OH)(CH₃), —CH₂—SH, —CH₂—CH₂—S—CH₃, —CH₂—Ar, wherein Ar is phenyl or p-phenol.

9. The method of claim 7, wherein the compound of formula (I) is selected from the group consisting of:

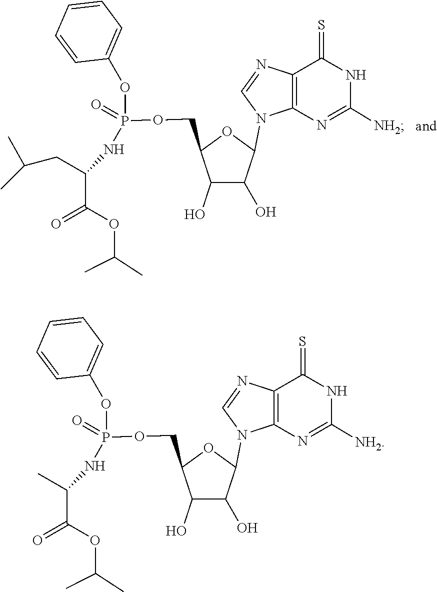

10. The method of claim 9, wherein the compound of formula (I) is:

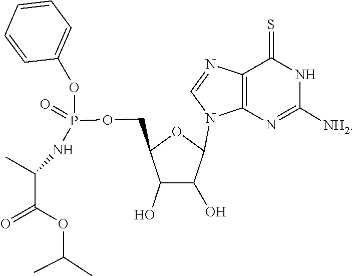 [SMILES: CC(C)OC(=O)[C@H](C)NP(=O)(OC[C@H]1OC(n2cnc3c(=S)[nH]c(N)nc32)C(O)C1O)Oc1ccccc1]

11. The method of claim 7, wherein the cancer is selected from the group consisting of acute lymphoblastic leukemia (ALL), chronic myeloid leukemia (CML), childhood acute lymphoblastic leukemia, acute promyelocytic leukemia. (APL), acute myeloid leukemia (AML), and chronic myelomonocytic leukemia (CMML).

12. The method of claim 11, wherein the cancer is acute lymphoblastic leukemia (ALL) or chronic myeloid leukemia (CML).

13. The method of claim 7, wherein the cancer is a soft tissue sarcoma.

14. The method of claim 13, wherein the soft tissue sarcoma is selected from the group consisting of a malignant peripheral nerve sheath tumor, a triton tumor, a rhabdomyosarcoma, and a synovial sarcoma.

15. The method of claim 7, wherein the autoimmune disease is an inflammatory bowel disease.

16. The method of claim 15, wherein the inflammatory bowel disease is Crohn's disease or ulcerative colitis.

17. The method of claim 7, wherein the autoimmune disease includes organ transplant rejection.

18. The method of claim 7, further comprising administering one or more additional therapeutic agents with the compound of formula (I).

19. The method of claim 18, wherein the disease is an inflammatory bowel disease and the one or more additional therapeutic agents is selected from the group consisting of mesalamine, prednisone, and infliximab.

20. The method of claim 18, wherein the disease is acute lymphoblastic leukemia (ALL) and the one or more additional therapeutic agents is selected from the group consisting of methotrexate, vincristine, cytarabine, cyclophosphamide, daunorubicin, and prednisone.
